(19) United States
(12) Patent Application Publication
(10) Pub. No.: US 2019/0345429 A1
(43) Pub. Date: Nov. 14, 2019

(54) Cell Holding Device for Microinjection

(71) Applicant: Lidong Qin, Houston, TX (US)

(72) Inventors: Lidong Qin, Houston, TX (US); Myeong Chan Jo, Houston, TX (US); Joon Hee Jang, Katy, TX (US); Yuan Ma, Toronto (CA)

(73) Assignee: Lidong Qin, Houston, TX (US)

(21) Appl. No.: 16/406,740

(22) Filed: May 8, 2019

Related U.S. Application Data
(60) Provisional application No. 62/668,606, filed on May 8, 2018.

(51) Int. Cl. C12M 1/32; C12N 15/89

(52) U.S. Cl. CPC C12M 23/12; C12N 15/89

(57) ABSTRACT

A device for holding a plurality of cells is provided. The device includes a top layer having a plurality of spaced apart microwells which are not in contact with each other, each microwell being open on top and sized to hold a single cell. Each microwell has a top cross sectional shape having a perimeter which includes at least one inner concave angle and at least one inner convex angle, for providing increased friction between the microwell and the cell contained within.

### CROSS-REFERENCES TO RELATED APPLICATIONS

This application claims priority from U.S. Provisional Application Ser. No. 62/668,606 filed May 8, 2018, which is hereby incorporated herein by reference in the respective in its entirety.

### TECHNICAL FIELD

This invention relates to the filed of biology, and more particularly, to devices for holding single cells for enabling experiments to be performed in the cells.

### BACKGROUND OF THE INVENTION

Delivery of molecules, DNAs and proteins into the cytoplasm of a cell is essential for cell-based research and development as well as clinical application such as drug screening and in vitro fertilization. Microinjection is one of the most precise techniques to inject cargo materials into the single cells or to remove cellular organelles. Motorized translation stages with high-resolution and high-precision in combination with microinjectors precisely control the position of injection needle, which punctate cell membrane and dispense predetermined volume of the cargo materials into the cells. The precision and controllability make the microinjection technique stand out from other delivery methods such as electroporation and viral transduction.

Microinjection techniques, however, requires well trained personnel. The success rate of injection highly depends to the operator's skill. Typically, it takes 15-30 s of operation time for microinjection per cell for a skilled operator. During the injection, even small movement will punctuate large hole on the cell membrane, which can lead to cell apoptosis.

### BRIEF SUMMARY OF EMBODIMENTS OF THE INVENTION

The inventors have found that restraining cell movement by a cell holding device during the injection can minimize damage to the cell membrane and to diminish the time required for successfully perform a microinjection.

To address the issues of current microinjection techniques, the present invention relates to a device configured to hold a plurality single cells in an array and restrain cell movement during injection. Traditional injection methods require to hold a cell manually with one arm and operate the injector with the other arm, followed by release of the cell. In the device of the present invention, microwells are provided such that each microwell holds a single cell. Each microwell is shaped to anchor the cell to the microwell and to restrain movement during injection and textured surface.

By using the device of the present invention, the need of cell holding arm is obviated. This makes microinjection process faster and less skill dependent. In some embodiments of the present invention, the microwells isolate the cells in pre-defined positions. This enables automation of injection and tracking of the individual cells.

An aspect of some embodiments of the present invention relates to a device for holding a plurality of cells, the device comprising a top layer having a plurality of spaced apart microwells which are not in contact with each other, each microwell being open on top and sized to hold a single cell. Each microwell has a top cross sectional shape having a perimeter which includes at least one inner concave angle and at least one inner convex angle, for providing increased friction between the microwell and the cell contained within.

In a variant, the at least one inner convex angle is acute.

In another variant, the at least one of the microwells has the top cross-sectional shape having the perimeter which includes a plurality of inner concave angles and a plurality of inner convex angle, such that a plurality of extensions are formed, protruding inward from the perimeter, to provide increased friction between the microwell and the cell contained within.

In some embodiments of the present invention, at least one of the inner convex angles is acute.

In yet another variant, the top layer is transparent to visible light.

In a further variant, the device further comprises a substrate bonded to a bottom of the top layer.

In a variant, the substrate is rigid.

In another variant, the substrate is made of material that is transparent to visible light.

In yet another variant, at least one of the microwells is open on both the top and bottom of the top layer, and the substrate closes the at least one of the microwells at the bottom of the top layer.

In a further variant, the microwells are arranged to form an array of rows parallel to each other and of rows parallel to each other.

Each row may be perpendicular to the columns.

In yet a further variant, the top layer comprises at least one depression having a floor. The at least one depression comprises at least one respective microwell notched on the floor.

In a variant, each depression comprises a plurality of respective microwells notched on the respective floor of the depression.

In another variant all microwells are notched on the floor of the at least one depression.

In yet another variant, the top layer comprises at least two depressions spaced apart from each other and not in communication with each other, each depression having a respective floor. Each depression comprises at least one respective microwell notched on the respective floor of the depression.

In a further variant, each depression comprises a plurality of respective microwells notched on the respective floor of the depression.

In yet a further variant, all microwells are notched on the floors the at least two depressions.

In some embodiments of the present invention, the device of some embodiments the present invention is shaped as a microscope slide.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1 is a side cross sectional view of a cell holding device with up-open microwells, according to some embodiments of the present invention;

FIG. 2 is a side cross sectional view of a cell holding device with microwells are open both on the top and bottom, and the bottoms of the microwells are closed by the substrate, according to some embodiments of the present invention;

FIG. 3 is an exploded view of a cell holding device of some embodiments of the present invention, having a plurality of larger wells, such that each well contains a group of spaced apart microwells;

FIG. 4 is a perspective view of a cell holding device of the present invention configured as part of a microscope slide;

FIGS. 5-7 are perspective views of cell holding device of the present invention configured as part of a microscope slide, in which the microwells are separated into different spaced apart groups;

FIGS. 8-11 illustrate different shapes in of the microwells, according to some embodiment of the present invention;

FIG. 12 illustrates a cell suspension added to the cell holding device of the present invention;

FIGS. 13-16 are photographs taken by a microscope at different magnifications, showing single cells captured by the microwells of the cell holding device of the present invention;

FIG. 17 illustrates the cell holding device of the present invention, used in conjunction with a microscope and a microinjector;

FIGS. 18-20 are photographs taken using a microscope, illustrating different stages of microinjection of material into a cell held inside a microwell of the cell holding device of the present invention;

FIGS. 21-27 illustrate steps of a method for fabricating a cell holding device according to some embodiments of the present invention;

FIG. 28 is a top view of a photomask used in the fabrication of the cell holding device, according to some embodiments of the present invention;

FIG. 29 is top view illustrating the completed cell holding device, according to some embodiments of the present invention; and

FIGS. 30-33 illustrates steps of a method for treating the networked cell-holder chip for adhesion of cells, according to some embodiments of the present invention.

It should be understood that the invention can be practiced with modification and alteration, and that the invention be limited only by the claims and the equivalents thereof.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

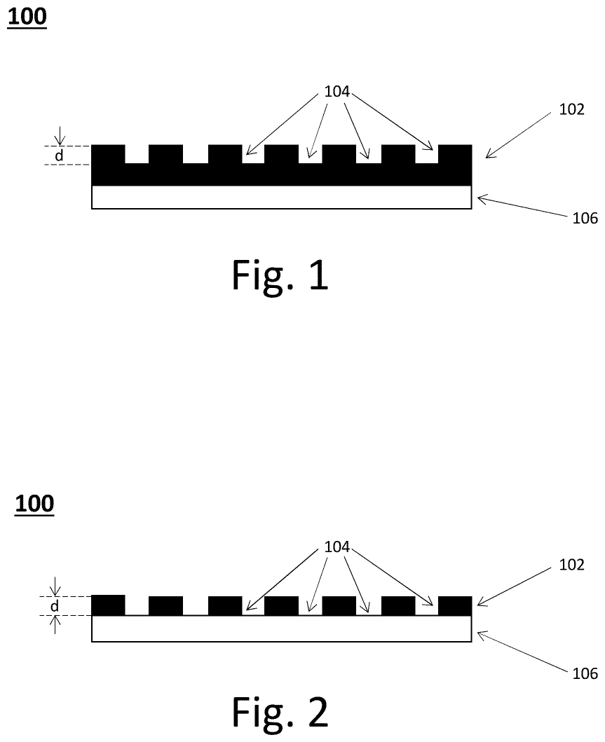

Referring now to the drawings, FIG. 1 is a side cross sectional view of a cell holding device 100 with up-open microwells 104, according to some embodiments of the present invention.

The cell holding device 100 includes a top layer 102 having a plurality of spaced apart microwells 104 which are open on top. Each microwell 104 has a depth d and is configured to hold an individual cell. Only a single cell is loaded into each microwell 104. The microwells 104 do not pose a spatial constraint to cell morphology changes for culturing. The microwells are separated from each other.

The top layer 102 includes biocompatible material and is transparent to visible light, such as PDMS (polydimethylsiloxane), PMMA (poly (methyl methacrylate)), PC (polycarbonate), PS (polystyrene), liquid silicon rubber (LSR), and room-temperature volcanizing (RTV) silicon, for example. In some embodiments of the present invention, the thickness of the top layer 102 is between 0.5 mm and 3 mm. In some embodiments of the present invention, the microwells are set to form an array of rows parallel to each other and of columns parallel to each other. Is some embodiments of the present invention, each row is perpendicular to the columns.

In some embodiments of the present invention, the top layer 102 is joined to a substrate 106. The substrate 106 is thin material that is transparent to visible light (such as glass or polyvinylchloride, polystyrene, polycarbonate, and/or cyclic olefin copolymer (COC) that is highly compatible for microscope, for example). In some embodiments of the present invention, the thickness of the substrate 106 is between 0.1 mm and 1.5 mm. The substrate 106 provides a rigid, flat base for supporting the top layer 102. The substrate 106 provides structural strength to the chip, and therefore makes the chip 100 easier to handle and move than the more compliant top layer 102.

In some embodiments of the present invention, as shown in FIG. 2 the microwells 104 of the top layer 102 are open both on the top and bottom. The bottoms of the microwells 104 are closed by the substrate 106.

The device 100 used for single cell isolation, single cell imaging, and single cell based assay applications, as the device 100 is configured to contain the single cells in culture wells during the observation and analysis.

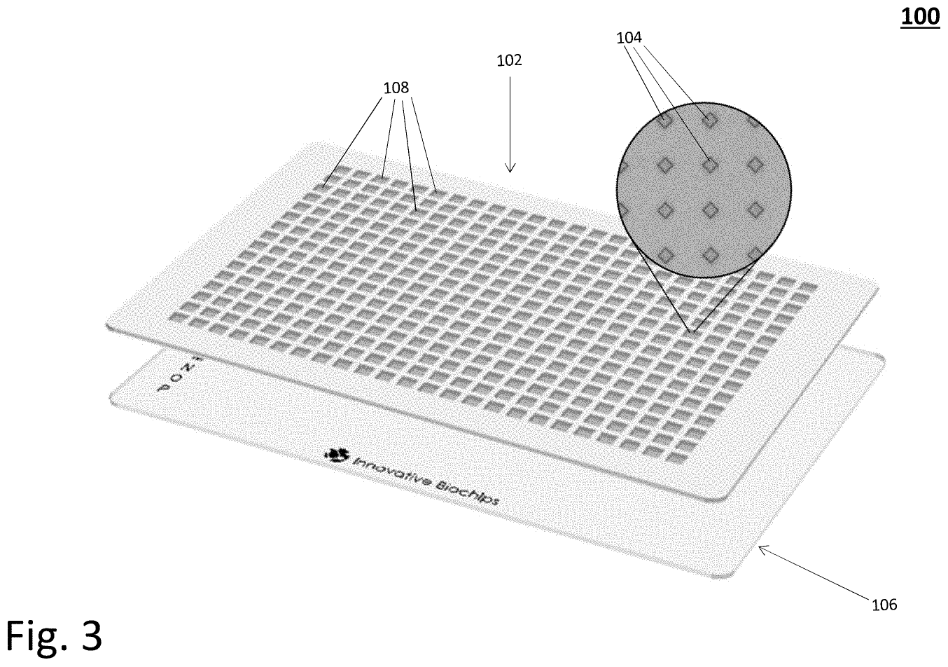

FIG. 3 is an exploded view of a cell holding device 100 of some embodiments of the present invention, having a plurality of spaced-apart depressions 108, such that each depression 108 contains a group of spaced apart microwells. In some embodiments of the present invention, microwells are located only in the depressions and not outside the depression.

The microwells which are in the same depression do not prevent crosstalk between the cells contained therein, as the medium in the depression is common to all cells in the microwells of the depression. Therefore, chemicals may be exchanged between cells via the medium.

On the other hand, there is atmospheric barrier between different depressions, preventing communication between cells located in different depressions. This is because the medium in one depression does not communicate with the media of other depressions. The purpose of multiple depressions is to handle multiple cell types or different media.

In some embodiments of the present invention, top layer 102 includes a plurality of spaced-apart depressions 108. Each depression 108 has a floor into which one or more microwells 104 are notched. This allows a researcher to place different kinds of cells in each depression, or place cells of the same kind in all depressions while to exposing cells in each depression to different materials. In some a non-limiting example, the depression is about 0.8 mm deep, the top layer is 1 mm deep, and each microwell has a depth of 10-20 μm. It should be noted that these sizes are merely examples, and the scope invention extends to different sizes as well.

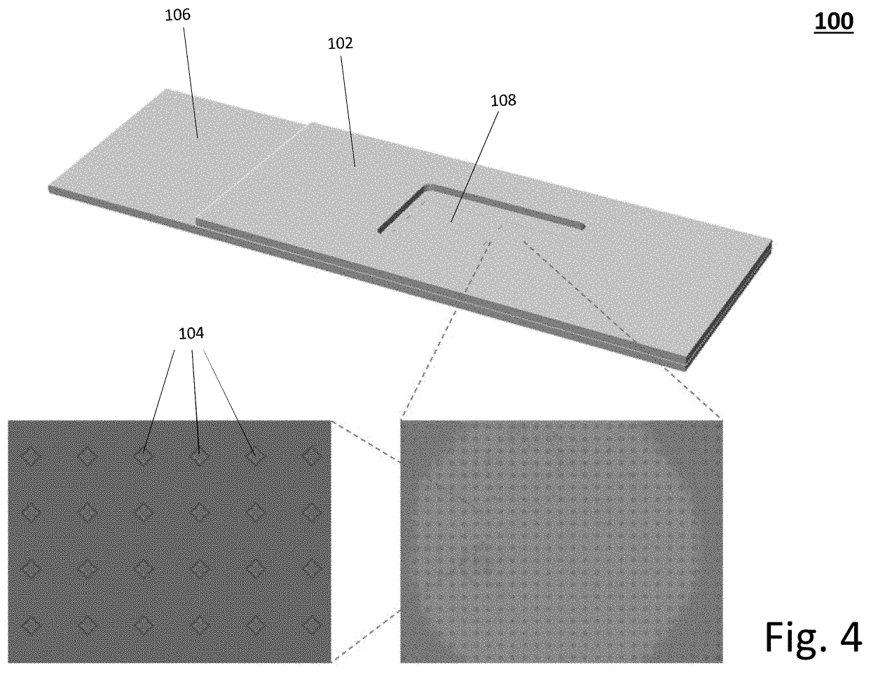

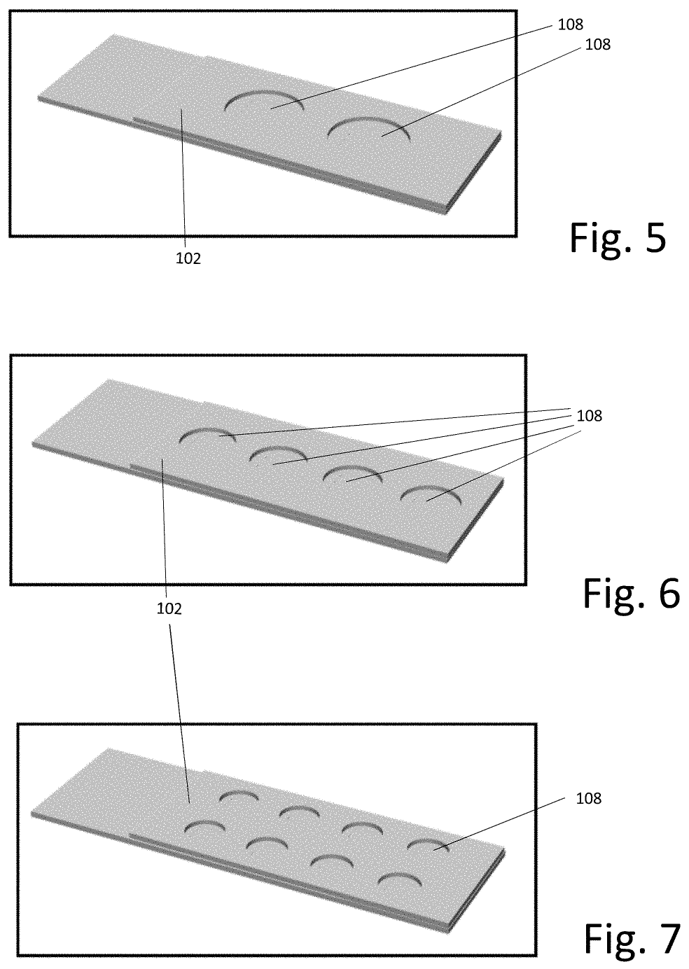

FIG. 4 is a perspective view of a cell holding device 100 of the present invention configured as part of a microscope slide. FIGS. 5-7 are perspective views of cell holding device of the present invention configured as part of a microscope slide, in which the microwells are located inside different spaced apart depressions 108.

In the example of FIG. 4, the top layer 102 and the substrate 106 are elongated. Optionally, the substrate 106 is wider than the top layer 102, to enable a user to handle the and move the device 100 without touching the top layer 102.

In some embodiments, the top layer 102 has a single depression 108 as described above with respect to the depressions 108 of FIG. 3 the microwells are located at the floor of the depression 108, as explained above. The depression enables the device 100 to hold a medium which provides sustenance to the cells in the microwells, as wells as to hold a solution which contains the cells, before the cells are captured in the microcells. In some embodiments of the present invention, the top layer includes a plurality of depressions 108, as explained above and shown in FIGS. 5-7.

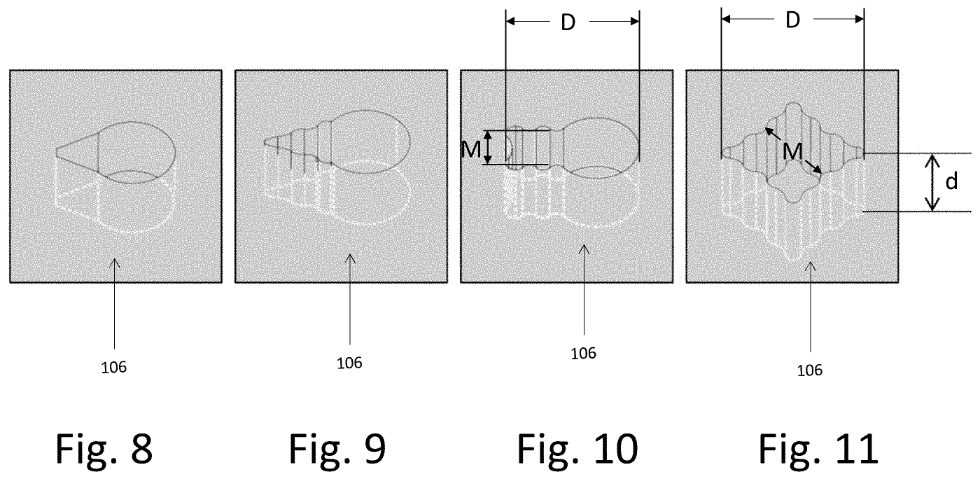

FIGS. 8-11 illustrate different shapes in of the microwells, according to some embodiment of the present invention.

In some embodiments of the present invention, the microwells 104 in the chip have walls are shaped to increase friction between the microwell and the cell contained within. This friction decreases the movement of the cell within the microwells and therefore facilitates microinjection in the cells.

In some embodiments of the present invention, each microwell, has a top cross-section having a perimeter which includes one or more inner concave angles and one or more inner convex angles. In some embodiments of the present invention, at least one of the convex angles is an acute angle. As will be explained further below, these shapes of the top cross-section are determined by the shape of the photomask used to fabricate the chip.

In some embodiments of the present invention, the perimeter of the top cross section has a plurality of inner concave angles and inner convex angles, such that a plurality of extensions protrude inwards from the perimeter, as seen, for example in FIGS. 8-11. Thanks to the advances in printing, the photomask has sub-micrometer spatial resolution. Thus, the complex shapes of the microwells can be directly printed on the photomask and the pattern is transferred to photoresistor.

The microwell 104 has two horizontal dimensions: a large horizontal dimension D and a small horizontal dimension M. The dimensions D and M may be perpendicular to each other and are similar to each other (e.g. within 50% of each other). Both dimensions D and M are slightly larger than a dimension of the cell, to allow the cell of be held in the microwells. A non-limiting example of a dimension D is between 10 μm and 20 μm.

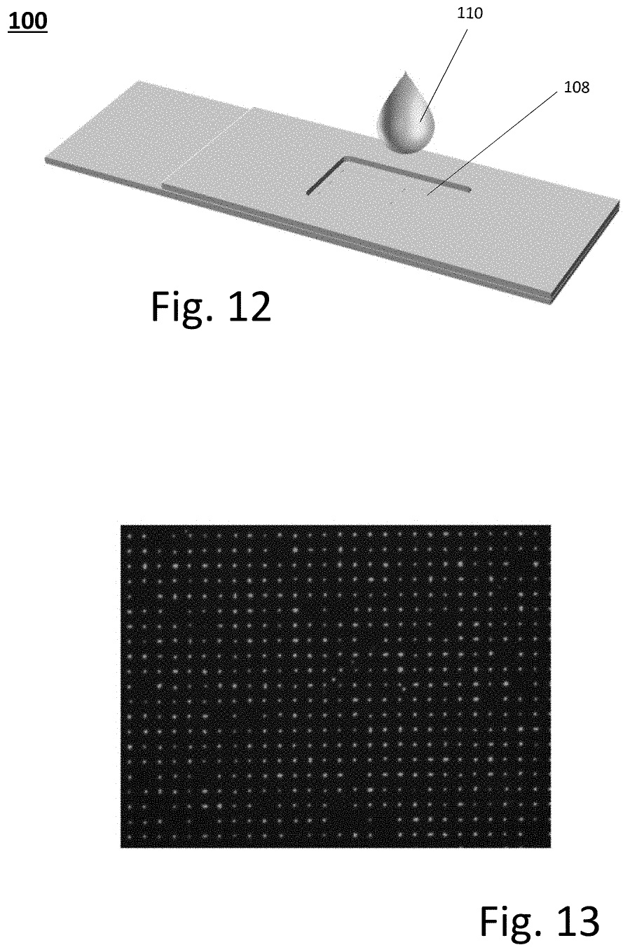

FIG. 12 illustrates a cell suspension 110 added to the cell holding device 110 of the present invention.

The cell suspension includes a plurality of the cells that are to be captured into the microwells. The cell suspension is dropped in depression 108, so that the cells within the device 100 are captured by the microwells. In a device produced by the inventors, about 1 mm of height the depression is sufficient to hold 100 μl of the cell suspension. The same height of the depression 108 also enables low angle approach of the injection needle as explain further below and shown in FIG. 10. In a device 100 manufactured by the inventors, most of the microwells were filled with the isolated single cells with fill factor of >90%. To speed up the capture of the cells in the microwells, the device 100 may be centrifugated, for example at a speed of 400 g for 1 min.

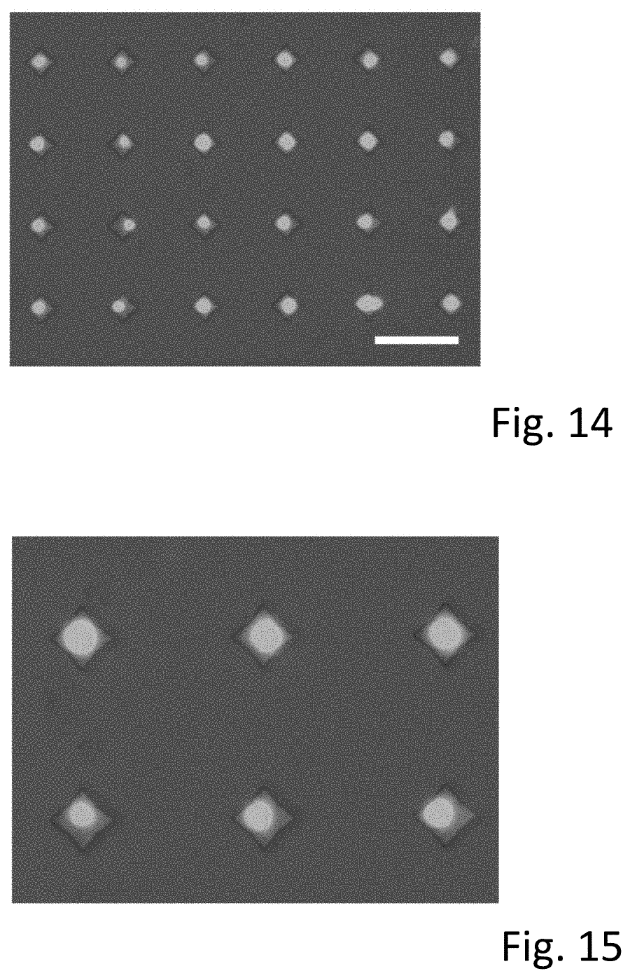

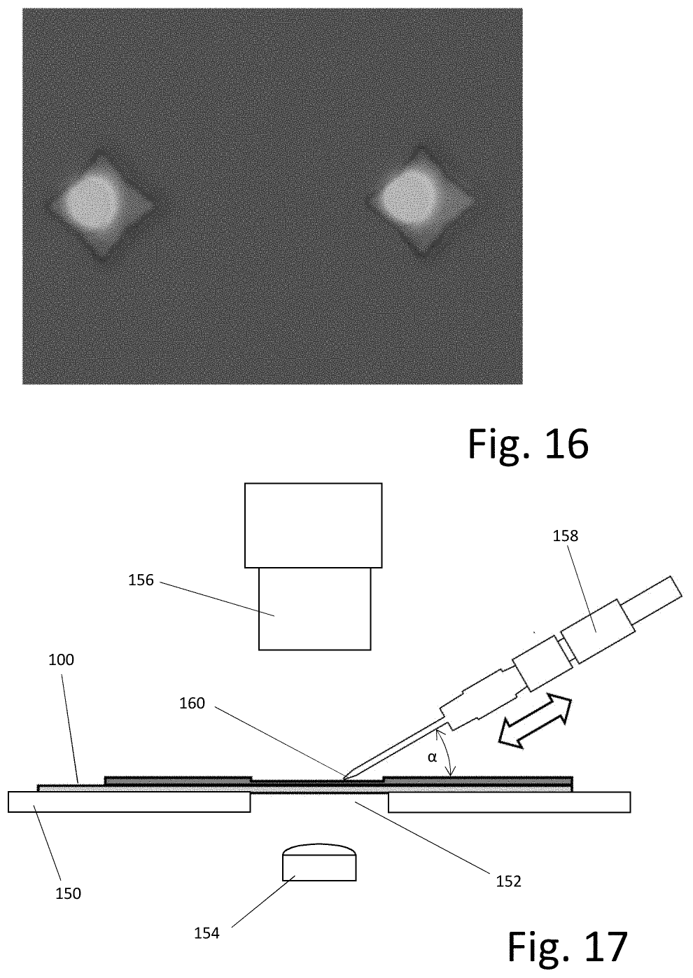

FIGS. 13-16 are photographs taken by a microscope at different magnifications, showing single cells captured by the microwells of the cell holding device of the present invention. The cells are shown in green.

FIG. 17 is a cross sectional view which illustrates the cell holding device 100 of the present invention, used in conjunction with a microscope and a microinjector.

The upright microscope has a holding plate 150 with an opening 152, a light source 154, and a magnifying lens 156. A lighting system directs light from the light source 154 through the opening 152, the device 100, to the lens 154. In some examples, the microscope may be configured as an inverted microscope, in which positions of the light source 154 and the magnifying lens 156 are inversed. A microinjector 158 has a needle tip 160 that is in the viewing region of the lens 156. This enables a user to view and move the microinjector to perform a microinjection in the cell.

The shape of the device 100 enables the needle of the microinjector to be set at a low angle a respect to the device 100. In a non limiting example, the angle a may be between 15 and 45 degrees. This enables easier control of the microinjector and also enables the user to use the needle of the microinjector to push the cells against the walls of their respective microcells.

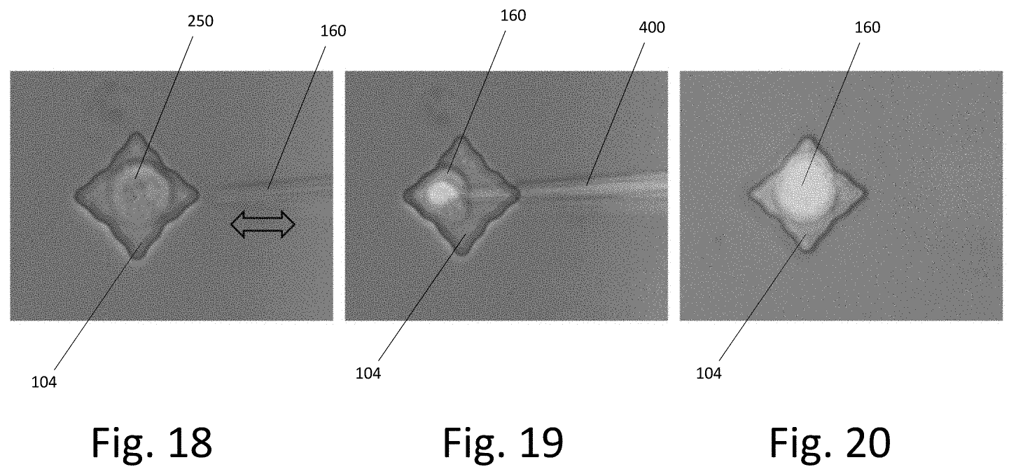

FIGS. 18-20 are photographs illustrating a microinjection performed on a cell 250 in a microwell 104 having a shape configured to increase friction between the microwell' s wall and the cell 250 contained in the microwell, according to some embodiments of the present invention.

In FIG. 18, the cell 250 is in the microwell 104 and the tip 160 of the microinjection needle is outside the microwell. In FIG. 19, the tip 160 needle gently pushes the cell 250 to a corner of microwell 104 and the gripping surface of the wall holds the cell 250 in position during injection by the friction between cell membrane and the wall of the microwell. Next, the tip 106 of injection needle advances to pierce the cell membrane and injects the designed amount of liquid into the cell 250. Lastly, in FIG. 20, the tip 400 needle is retrieved and the successful injection can be confirmed in the fluorescence of the cell 250.

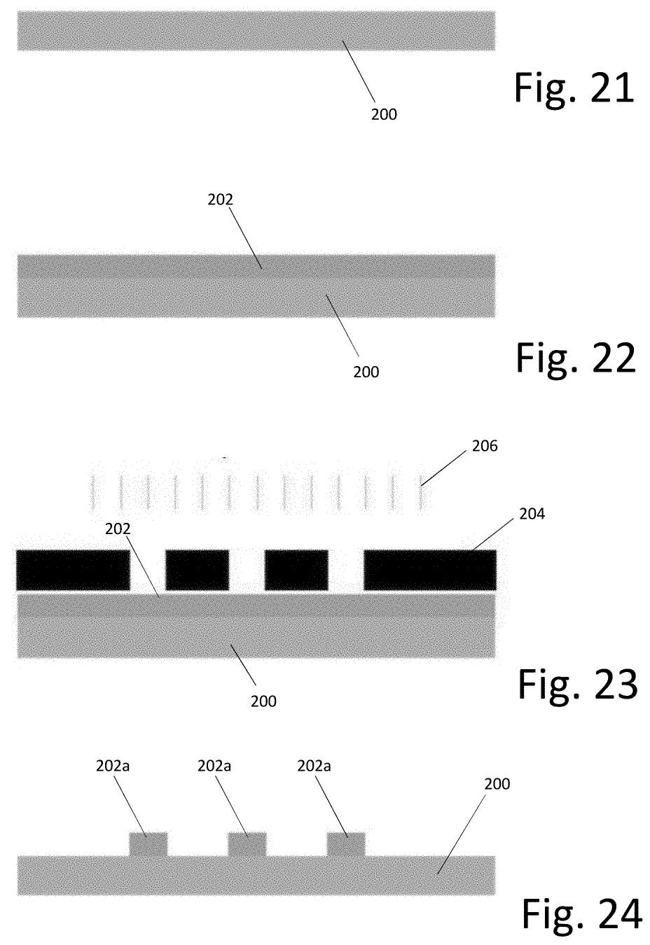

FIGS. 21-27 illustrate steps of a method for fabricating a cell holding device according to some embodiments of the present invention.

The structure of the chip is first plotted with computer-aided design (CAD) program and then a photomask 204 is created by printing the CAD design on glass or quartz substrate. The CAD design plotted on photomask will be transferred to photoresistor layer, as will be explained further below.

In FIG. 21, a wafer 200 is provided. The wafer 200 may be, for example, a silicon wafer. In FIG. 22 a photoresistor layer 202 is laid on the wafer 200. The photoresistor may include, for example SU-8 photoresist. The SU-8 photoresist may be spin-coated onto the silicon wafer at 500 rpm for 10 seconds with an acceleration of 100 rpm/s, then at 3,000 rpm for 30 s with an acceleration of 300 rpm/s. The wafer 200 may be then soft-baked, for example for about 10 minutes at about 95° C. The depth of the microwells is determined by the photoresist thickness. The spin coating and baking parameters can be varied to achieve desired thickness.

In FIG. 23, the photomask 204 is placed above photoresistor layer 202 and light 206 emitted from a light source is shined at the photoresistor layer 204 though the photomask 204. Sections 202a of photoresistor that are exposed to the light 206 harden, while those that are not exposed to the light 206 are washed away via a developer, as seen in FIG. 24. In a variant, the photoresistor layer is exposed to UV light of 200 mJ/cm² for 6 seconds, and post-exposure baking is performed immediately after for 1 minute at 65° C. and the for 4 minutes at 95° C. In a variant, the photoresistor is then developed in the developer for about 6 minutes, and then the wafer and photoresistor are hard-baked for 30 minutes at 150° C.

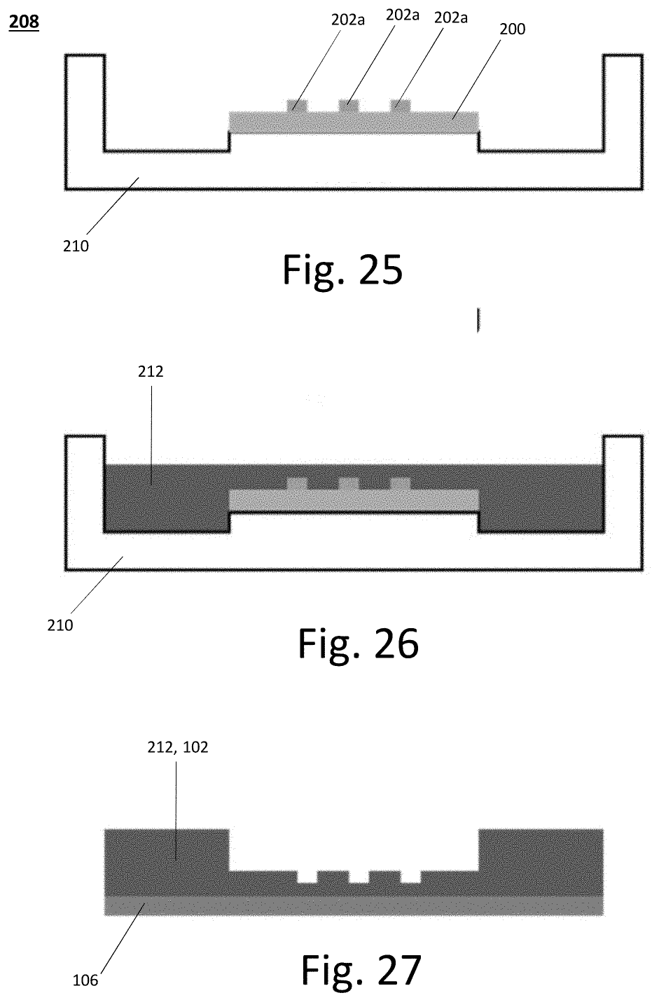

The hard-baked wafer 200 and developed photoresistor 202a serve as a mold 208, shown in FIG. 25. The mold 208 incudes a basin 210 and wafer 200 joined to the developed photoresistor 202a. The basin 210 is configured for receiving and holding a liquid. The wafer 200 is joined to the inner base of the basin 210, such that the developed photoresistor sections 202a are on the side of the wafer 200 that is opposite to the side of the wafer that contact the basin 210.

In FIG. 26, a liquid 212, which will ultimately form the top layer 102 of FIGS. 1-3, is poured into the basin 210. The liquid 212 is poured in a quantity that enables the top layer to have a desired height. Depending on the desired height of the liquid, the developed photoresistor sections 202a may be fully covered by the liquid 212, or may partially protrude above the surface of the liquid 212. The liquid 212 is manipulated to harden into an elastic form and is then peeled off from the basin 210 to from the top layer 102, as seen in FIG. 27. The top layer 102 is the joined to the substrate 106.

In some embodiments of the present invention, the liquid 212 is a 10:1 mixture of a PDMS oligomer with a crosslinking prepolymer of the PDMS agent from a Sylgard™184 kit. The mixture is placed under vacuum for degassing, and is the poured into the basin 210 of the mold 208. The mixture is cured at 80° C. for 2 hours inside the mold 208 to assume a solid form. Once solid, the mixture is peeled off from the mold. Oxygen plasma is applied to the upper layer 102 and the thin substrate 106, and then the upper layer 102 and the thin substrate 106 are bonded together. Finally, the bottomless well plate is integrated to the bonded upper layer 102.

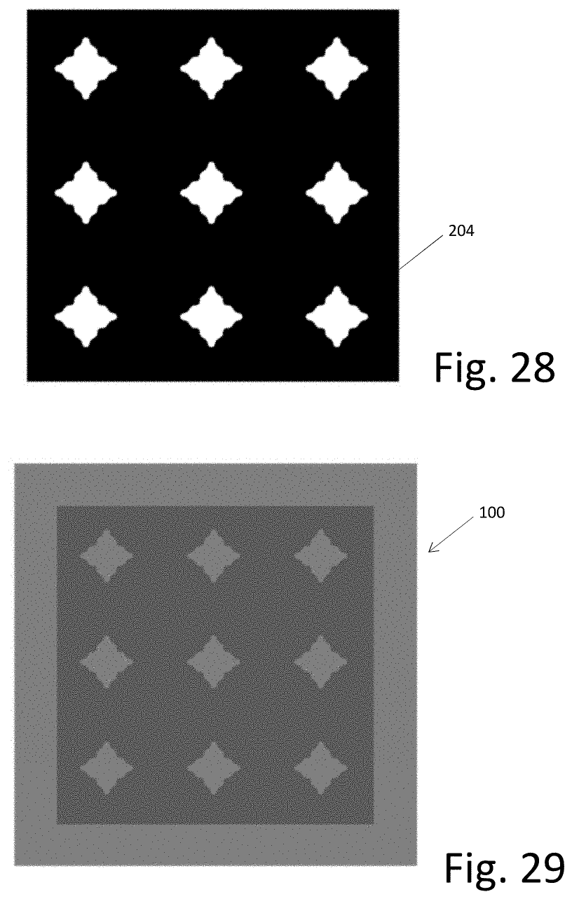

FIG. 28 is a top view of a photomask 204 used in the fabrication of the cell holder device, according to some embodiments of the present invention. FIG. 29 is top view illustrating the completed networked cell holder device 100, according to some embodiments of the present invention.

FIGS. 30-33 illustrates steps of a method for treating the networked cell holder device 100 for adhesion of cells, according to some embodiments of the present invention.

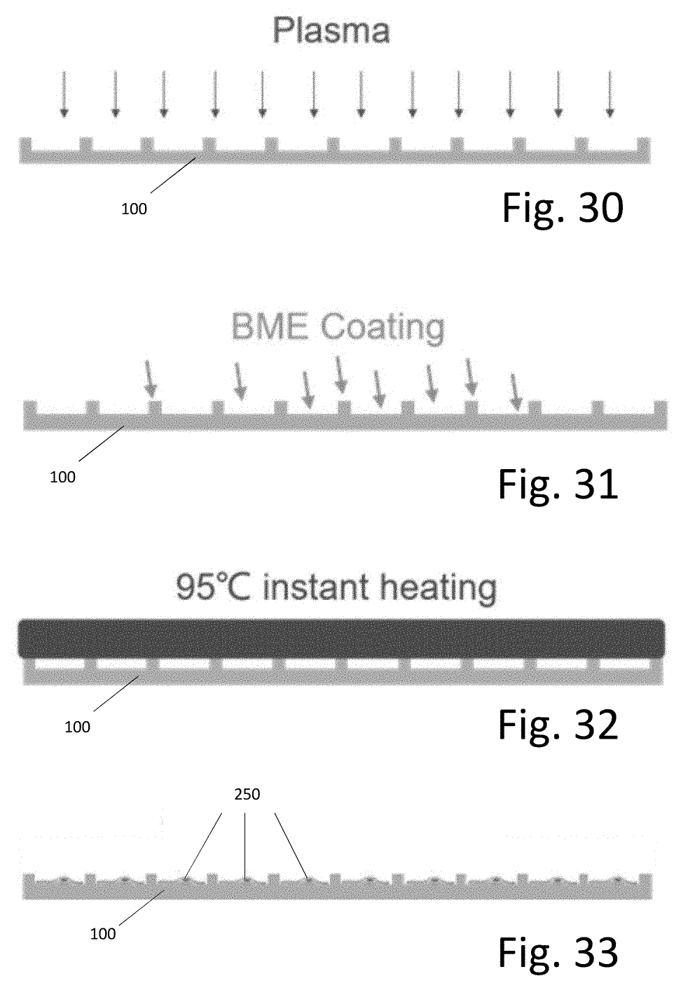

In FIG. 30, the surface of the device 100 is treated with oxygen plasma (1 min at an oxygen flow rate of 20 SCCM, a chamber pressure of 500 mtorr, and a power of 50 W). In FIG. 31, the surface of the chip 100 is covered with a droplet of Basement Membrane Extract (BME) and placed at 37° C. for 1 hour. After coating, the excessive liquid BME is removed. In FIG. 32, the chip 100 is moved onto a 95° C. digital dry bath (Bio-Rad) for 1 second to denature the BME coated on the surface of the chip 100. After the heating in the digital dry bath, invisible tape is used to peel off the excessive BME. In FIG. 33, cells 250 are introduced in the microwells of the chip 100.

What is claimed is:

1. A device for holding a plurality of cells, the device comprising:
a top layer having a plurality of spaced apart microwells which are not in contact with each other, each microwell being open on top and sized to hold a single cell;
wherein each microwell has a top cross sectional shape having a perimeter which includes at least one inner concave angle and at least one inner convex angle, for providing increased friction between the microwell and the cell contained within.

2. The device of claim 1, wherein the at least one inner convex angle is acute.

3. The device of claim 1, wherein the at least one of the microwells has the top cross-sectional shape having the perimeter which includes a plurality of inner concave angles and a plurality of inner convex angle, such that a plurality of extensions are formed, protruding inward from the perimeter, to provide increased friction between the microwell and the cell contained within.

4. The device of claim 3, wherein at least one of the inner convex angles is acute.

5. The device of clam 1, wherein the top layer is transparent to visible light.

6. The device of claim 1, further comprising a substrate bonded to a bottom of the top layer.

7. The device of claim 6, wherein the substrate is rigid.

8. The device of claim 6, wherein the substrate is made of material that is transparent to visible light.

9. The device of claim 6, wherein at least one of the microwells is open on both the top and bottom of the top layer, and the substrate closes the at least one of the microwells at the bottom of the top layer.

10. The device of claim 1, wherein the microwells are arranged to form an array of rows parallel to each other and of rows parallel to each other.

11. The device of claim 10, wherein each row is perpendicular to the columns.

12. The device of claim 1, wherein:
the top layer comprises at least one depression having a floor; and
the at least one depression comprises at least one respective microwell notched on the floor.

13. The device of claim 12, wherein each depression comprises a plurality of respective microwells notched on the respective floor of the depression.

14. The device of claim 12, wherein all microwells are notched on the floor of the at least one depression.

15. The device of claim 12, wherein:
the top layer comprises at least two depressions spaced apart from each other and not in communication with each other, each depression having a respective floor; and
each depression comprises at least one respective microwell notched on the respective floor of the depression.

16. The device of claim 15, wherein each depression comprises a plurality of respective microwells notched on the respective floor of the depression.

17. The device of claim 15, wherein all microwells are notched on the floors the at least two depressions.

18. The device of claim 6, shaped as a microscope slide.
